(19) United States
(12) Patent Application Publication
(10) Pub. No.: US 2008/0193482 A1
(43) Pub. Date: Aug. 14, 2008

(54) METHOD OF CANCER SCREENING; METHOD OF CANCER TREATMENT; AND METHOD OF AUTO-IMMUNE DISEASE TREATMENT

(76) Inventor: John R. Woodward, Dallas, TX (US)

Correspondence Address: Michael A. O'Neil;Michael A. O'Neil, P. C., Suite 820, 5949 Sherry Lane, Dallas, TX, 75225, US

(21) Appl. No.: 12/100,089

(22) Filed: Apr. 9, 2008

Related U.S. Application Data
(63) Continuation-in-part of application No. 11/533,805, filed on Sep. 21, 2006.
Application No. 11/533,805 is a continuation of application No. 11/133,838, filed on May 19, 2005, now Pat. No. 7,125,836.
Application No. 11/133,838 is a division of application No. 11/032,399, filed on Jan. 10, 2005, now abandoned.
Application No. 11/032,399 is a continuation-in-part of application No. 11/003,293, filed on Dec. 3, 2004, now abandoned.
Application No. 11/003,293 is a continuation-in-part of application No. 10/946,213, filed on Sep. 21, 2004, now abandoned.

(51) Int. Cl. A61K 39/29; A61K 49/00; A61P 35/00; A61K 39/00

(52) U.S. Cl. 424/227.1; 424/9.2; 424/184.1

(57) ABSTRACT

A method of cancer screening comprising the steps of administering the Blood CA 27,29 testing procedure; if the result is positive administering a mammogram; if the result is positive administering a needle biopsy; if the result is positive administering a PET scan; if the result is positive administering a blood tumor cell count. If all of the foregoing steps are positive, the cancer is treated by selecting one or more treatments from a group of provided treatment according to the patient's body and condition. A method of treating auto-immune diseases comprises selecting one or more treatments from another group of provided treatments, the one or more treatments selected and administered according to the patient's body and condition.

### CROSS-REFERENCE TO RELATED APPLICATIONS

This application is a continuation-in-part application of application Ser. No. 11/533,805 filed Sep. 21, 2006, currently pending, the entire contents of which are incorporated herein by reference; which is a continuation application of application Ser. No. 11/133,838 filed May 19, 2005, now U.S. Pat. No. 7,125,836, the entire contents of which are incorporated herein by reference; which is a divisional of application Ser. No. 11/032,399 filed Jan. 10, 2005, abandoned, the entire contents of which are incorporated herein by reference; which is a continuation-in-part application of application Ser. No. 11/003,293 filed Dec. 3, 2004, currently pending, the entire contents of which are incorporated herein by reference; which is a continuation-in-part application of application Ser. No. 10/946,213 filed Sep. 21, 2004, currently pending, the entire contents of which are incorporated herein by reference.

### TECHNICAL FIELD

This invention relates generally to the detection and treatment of cancers and auto-immune diseases, and more particularly to a method of screening for and a method of treating duct cell cancer of the breast, squamous cell cancer of the uterine cervix, anal cancer, and auto-immune diseases including diabetes, multiple sclerosis, rheumatoid arthritis metabolic syndrome, lupus, and Acquired Immune Deficiency Syndrome (AIDS).

### BACKGROUND AND SUMMARY OF THE INVENTION

As is well known, various technologies are available to the medical profession for use in determining the presence of cancers in patients. Included are x-ray studies, magnetic resonance imaging (MRI) studies, CT scans, as well as studies of various body fluids such as blood, urine, etc. Potential sites for colon cancer, for example, can be investigated utilizing electro-optical technologies. In some cases needle biopsy or exploratory surgery is necessary to confirm either the presence or absence of suspected cancer.

Various techniques for treating cancers are also well known. Certain cancers can be surgically removed, whereas other cancers require radiation therapy, chemotherapy, or combinations of radiation therapy and chemotherapy. Other cancers are susceptible to control using one or more drug therapies.

Auto-immune diseases such as diabetes, multiple sclerosis, rheumatoid arthritis metabolic syndrome, lupus, and Acquired immune deficiency syndrome (AIDS) affect persons of all age, race, and gender. Heretofore, treatments available for auto-immune diseases have only been able to control each disease and any corresponding systems. Such treatments vary according to each disease, but typically comprise a combination of a number of medications, testing and diagnostic procedures, and/or various physical therapy treatments. Such treatments and corresponding research focus on cell replacement rather than cell death.

Cell death is generally accepted as a consequence of either a passive, degenerative process called necrosis, or of an active process called apoptosis. Apoptosis may be described as programmed cell death actively driven by the cell. Necrosis is a passive consequence of gross injury to the cell. Causative agents, which initiate cell death cycles, are a group of viruses named recombinant Adeno-Associated viruses (rAA viruses). This family of viruses causes the usual sequence of DNA signals to messenger RNA to reverse itself leading to alteration of immune response in either a positive or negative manner. An over-response leads to an auto-immune disease and an under-response results in rapid proliferation of cells during which the body loses its ability to control cell death, such as in the case of cancer.

The present invention comprises a method of cancer screening, a method of cancer treatment, a method for treatment of diabetes, and a method for treatment of auto-immune diseases which has proven successful in controlling epidermal cancers including, but not limited to, duct cell breast cancer, cervical squamous cancer, and anal cancer, and controlling auto-immune diseases including multiple sclerosis, diabetes, rheumatoid arthritis, metabolic syndrome, lupus, and Acquired Immune Deficiency Syndrome (AIDS). In accordance with the broader aspects of the invention, a method of cancer screening involves a series of testing procedures each more expensive than the one before. Only when results of each of the testing procedures are positive is the presence of cancer confirmed. The invention further comprises a method of treating cancer and insulin dependent Type I diabetes wherein the drug imiquimod is administered transdermally in conjunction with a vaccine that induces production of tumor necrosis factor, for example the BCG vaccine, and valacyclovia hydrochlorine tablets. The method of the present invention treats Type I diabetes by enabling the body to regenerate islet cells. Similarly, multiple sclerosis is managed and treated by enabling the body to repair nerve endings and regenerate damaged fibers.

### BRIEF DESCRIPTION OF THE DRAWING

A more complete understanding of the present invention may be had by reference to the following Detailed Description when taken in connection with the accompanying Drawings, wherein:

FIG. 1 is a flowchart illustrating initial steps in the cancer screening method of the present invention;

FIG. 2 is a flowchart illustrating subsequent steps in the cancer screening method of the present invention;

FIG. 3 is a flowchart illustrating the diabetes treatment method of the present invention;

FIG. 4 is a flowchart illustrating a screening method and cancer treatment comprising another embodiment of the present invention;

FIG. 5 is a flowchart illustrating another method of cancer treatment comprising yet another embodiment of the present invention; and

FIG. 6 is a flowchart illustrating an auto-immune disease treatment comprising still another embodiment of the present invention.

### DETAILED DESCRIPTION

### Introduction

The following examples describe a method of detecting and treating duct cell breast cancer, and a method for treating Type I diabetes. However, the present invention is equally applicable to other epidermal cancers, such as squamous cancer of the uterine cervix and anal cancer, and the treatment of and management of auto-immune diseases including multiple sclerosis, rheumatoid arthritis, metabolic syndrome, lupus, and AIDS.

### EXAMPLE

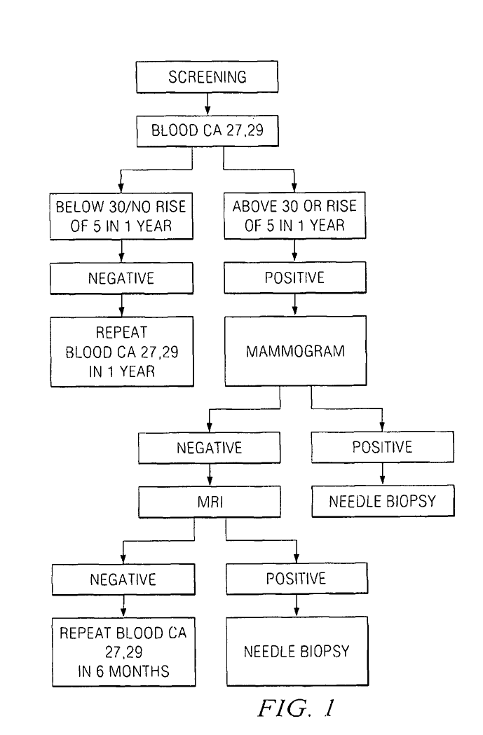

Referring to the Drawings, and particularly to FIG. 1 thereof, the early steps in the method of cancer screening of the present invention are shown therein. Screening begins with administration of the testing procedure known as Blood CA 27,29. The Blood CA 27,29 testing procedure has heretofore been utilized in monitoring the results of existing cancer treatment procedures. However, the Blood CA 27,29 procedure has not heretofore been used for cancer screening.

If the number comprising the results of the Blood CA 27,29 procedure is less than 20, and if there has been no increase in the number comprising the result of the Blood CA 27,29 testing procedure of 5 or more in the immediately preceding year, the result of the Blood CA 27,29 testing procedure is considered to be negative. The patient is then scheduled for follow-up testing utilizing the Blood CA 27,29 procedure in one year.

If the number comprising the result of the Blood CA 27,29 procedure is 30 or above, or if there has been an increase of 5 or more in the number comprising the result of the CA 27,29 testing procedure in the immediately preceding year, the result of the Blood CA 27,29 procedure is considered to be positive. In that event a mammogram testing procedure is administered. If the result of the mammogram testing procedure is negative, an MRI testing procedure is administered. If the result of the MRI testing procedure is negative, both the mammogram testing procedure and the Blood CA 27,29 testing procedure are re-administered in six months time. Conversely, if either the mammogram testing procedure is positive or the MRI testing procedure is positive, a needle biopsy of the identified lesion is performed.

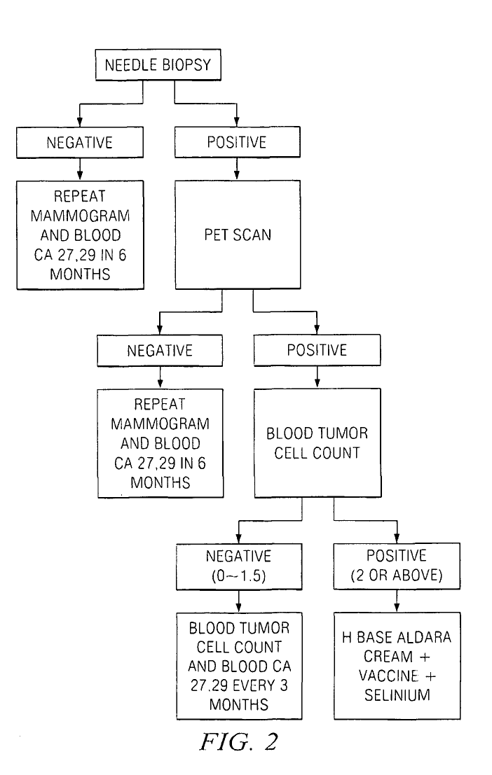

Referring to FIG. 2, if the results of the needle biopsy testing procedure are negative, both the mammogram testing procedure and the Blood CA 27,29 testing procedure are re-administered in six months. If the needle biopsy testing procedure is positive, a positron emission tomography (PET) scan testing procedure is administered.

If the result of the PET scan testing procedure is negative, the mammogram testing procedure and the Blood CA 27,29 testing procedure are re-administered in six months. If the result of the PET scan testing procedure is positive, a blood tumor cell count testing procedure is administered. If the result of the blood tumor cell count testing procedure is negative, that is, if the number comprising the result of the blood tumor cell count testing procedure is between 0 and 1.5, the blood tumor cell count testing procedure and the Blood CA 27,29 testing procedure are administered at three month intervals. Conversely, if the blood tumor cell count testing procedure is positive, that is, if the number comprising the result of the blood tumor cell count testing procedure is two or above, the cancer treatment procedure of the present invention is administered.

The cancer treatment procedure of the present invention comprises the transdermal administration of the drug imiquimod combined with a vaccine that induces production of tumor necrosis factor and administration of valacyclovia hydrochlorine tablets. Imiquimod is commercially available from 3M Pharmaceuticals under the trademark ALDARA® (imiquimod). A healthy human body produces the protein interferon alpha in response to an infection. Interferon alpha works to coat the infection or virus in order to make the infection or virus vulnerable to the human immune system. Imiquimod cream induces the production of interferon alpha once inside the human body, providing the needed interferon alpha not adequately produced by the patient's own body. In accordance with the present invention, ALDARA® (imiquimod) cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The mixture of imiquimod and H base cream as described above is administered transdermally, preferably by mixing ¼ cc ALOARA® (imiquimod) 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA® (imiquimod) 5% cream and H base cream is applied daily.

Tumor necrosis factor (also called TNFa, cachexin, or cachetin) stimulates T-cells that coordinate the immune system. In a healthy human body, tumor necrosis factor is released by white blood cells and other tissues in response to damage caused by an infection. Tumor necrosis factor is found in several vaccines. The preferable vaccine to be used in accordance with the present invention is the BCG vaccine, which is a common vaccine for tuberculosis, given in the United States and around the world. The tumor necrosis factor produced by the BCG vaccine and imiquimod cream work together to stimulate the T-cells and coordinate and improve the patient's immune system. Interferon alpha coats the virus or infection in order to make the virus or infection vulnerable to the tumor necrosis factor.

In accordance with the present invention, the vaccine is given in doses of 0.1 mL (100 μg) once every three weeks as long as the treatment continues. Valacyclovia hydrochlorine tablets, available from GlaxoSmithKline under the trademark VALTREX® (valacyclovir), is a drug commonly used in the treatment for genital herpes. In accordance with the present invention, VALTREX® (valacyclovir) tablets are consumed twice daily in 500 mg doses. The combination of the ALDARA® (imiquimod) and H base cream, the BCG vaccination, and the VALTREX® (valacyclovir) tablets is administered until a blood tumor cell count indicates that there are no cancer cells in the blood and a subsequent blood tumor cell count verifies a normal cell count and no mestastases are present. The results of the procedure are periodically monitored utilizing the Blood CA 27,29 testing procedure.

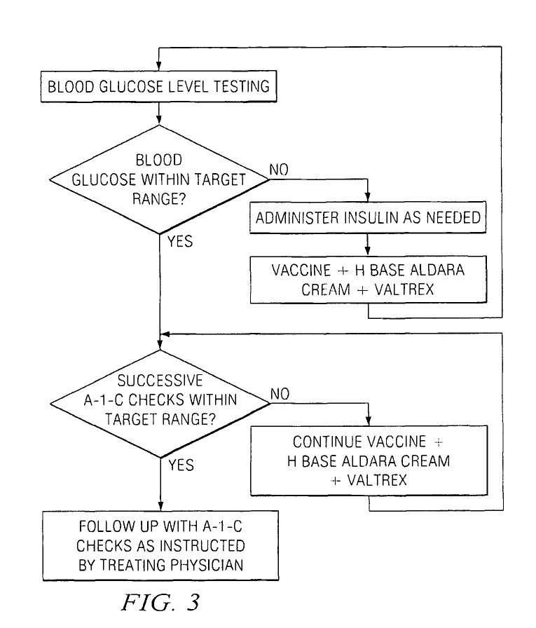

Referring to FIG. 3 thereof, persons with Type I diabetes must check their blood glucose levels at multiple intervals as directed by their physician. The average fasting blood glucose level should be between 70 mg/dL and 110 mg/dL. If the blood glucose level is not within the target level, the level must be corrected by taking insulin.

The diabetes treatment procedure of the present invention comprises the transdermal administration of the drug imiquimod combined with a BCG vaccination and administration of valacyclovia hydrochlorine tablets. Imiquimod is commercially available from 3M Pharmaceuticals under the trademark ALDARA® (imiquimod). A healthy human body produces the protein interferon alpha in response to an infection. Interferon alpha works to coat the infection or virus in order to make the infection or virus vulnerable to the human immune system. Imiquimod cream induces the production of interferon alpha once inside the human body, providing the needed interferon alpha not adequately produced by the patient's own body.

In accordance with the present invention, ALDARA® (imiquimod) cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The mixture of imiquimod and H base cream as described above is administered transdermally, preferably by mixing ¼ cc ALDARA® (imiquimod) 5% cream with ¼ cc H base cream and applying the resulting mixture to various locations, i.e., the inner thigh, abdomen, hip, arms, etc., of the patient. Various sites of administration prevent any possible skin irritation. The foregoing amount of the mixture of ALDARA® (imiquimod) 5% cream and H base cream is applied daily.

The results of the procedure are monitored utilizing blood sugar meters and a diary to record ongoing blood sugar levels. Additionally, A-1-C checks track the patient's overall blood sugar levels over two to three month periods, and is the most effective way to track long-range success of the treatment. The treatment method regenerates islet cells, which produce insulin. Once the patient no longer depends on insulin to correct blood sugar levels, the treatment continues until the patient has two or more sequential A-1-C checks in the target range, depending on the judgment of the treating physician. Once treatment is discontinued, A-1-C checks continue, but at less frequent intervals as recommended by the treating physician.

FIG. 3A illustrates an alternative treatment protocol which is similar in some respects to that of FIG. 3.

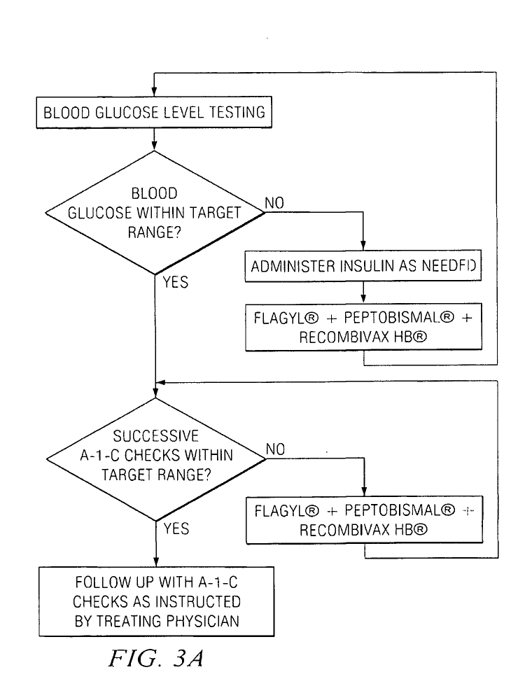

Referring to FIG. 4, there is shown a method of treatment for breast cancer comprising another embodiment of the present invention. A patient suspected of having cancer is screened according to the Blood CA 27,29 screening described herein in conjunction with FIG. 1. If the number comprising the results of the Blood CA 27,29 procedure is less than 30, and if there has been no increase in the number comprising the result of the Blood CA 27,29 testing procedure of ten (10) or more in the immediately preceding year, the result of the Blood CA 27,29 testing procedure is considered to be negative. The patient is then scheduled for follow-up testing utilizing the Blood CA 27,29 procedure in one year.

If the number comprising the result of the Blood CA 27,29 procedure is 30 or above, or if there has been an increase of 5 or more in the number comprising the result of the CA 27,29 testing procedure in the immediately preceding year, the result of the Blood CA 27,29 procedure is considered to be positive. In that event the patient begins medicinal treatment selected from one or more of the following medications and treatments: 1) oral administration of one tablet of about 10 mg to about 20 mg per day of ACOMPLIA™ (rimonabant), a Cannabinoid receptor type 1 (CB1) blocker; 2) Oral administration of calcitriol or 1,25-dihydroxycholecalciferol, which is the active form of vitamin D3 found in the body, taken in capsules of about 400 IU to about 800 IU once or twice daily; 3) administration of a RECOMBIVAX HB® (recombinant) Hepatitis B vaccine, administered once per month for three months followed by a booster vaccine six months after the final vaccine, each dose comprising about ¾ cc to about 1 cc; 4) following a low protein diet; 5) maintaining a directed exercise plan; 6) topical administration of about ¾ cc to about 1¼ cc daily of a cream mixture comprising ALDARA® (imiquimod) 5% cream and either H base cream or Q base cream as described herein in conjunction with FIG. 3; 7) administration of a combination of ULTRAM ER™ (tramadol) consumed in doses of about 50 mg to about 100 mg daily, according to each patient's needs, and VALTREX® (valacyclovir) consumed twice daily in doses of about 500 mg; 8) a combination of FLAGYL® (metronidazole) once or twice a day in a dose of about 250 mg to about 500 mg, one to two tablets of about 20 mg to about 40 mg per day of NEXIUM® (esomeprazole) per day, and one tablet of about 400 mg of AVELOX® (moxifloxacin) per day; 9) administration of the BCG vaccine, tuberculin; and 10) oral administration of three to four 1 g capsules of Omega 3 fatty acids two times daily, preferably Lovaza® brand fish oil pills. The patient's doctor may prescribe any combination of one or more of the above treatments according to the patient's medical history, physical condition, prognosis, and severity of illness. The selection of, dosage, and frequency of any treatment is determined and adjusted by the patient's doctor according to the patient's body. The triple therapy comprising a combination of FLAGYL® (metronidazole), NEXIUM® (esomeprazole), and AVELOX® (moxifloxacin) may be administered beginning one month after starting the combination of treatments selected by the doctor and may be continued for two weeks thereafter.

After a course of the patient's treatment as directed by the patient's doctor, a biopsy is performed on one or more suspicious tissue samples taken from the patient, either a needle biopsy of a tissue sample or an excisional biopsy of an entire lump or suspicious area, determined according the patient's individual prognosis. If the results of the biopsy is positive, the patient is given a series of RECOMBIVAX HB® (recombinant) Hepatitis 2 vaccines. Upon completion of treatment with RECOMBIVAX HB® (recombinant) positron emission tomography (PET) and/or computed axial tomography (CAT) scans are performed on as much of the patient's entire body as possible according to the patient's medical history and condition. If the results of the PET or CAT scans are positive the patient resumes the treatment plan comprising one or more of the above enumerated treatments and medications.

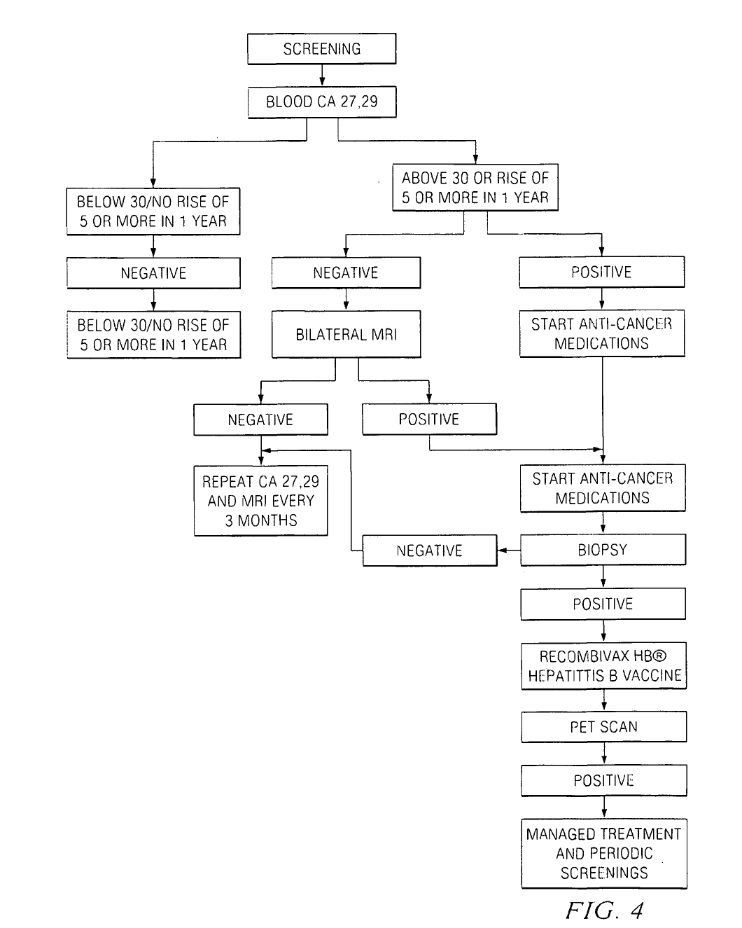

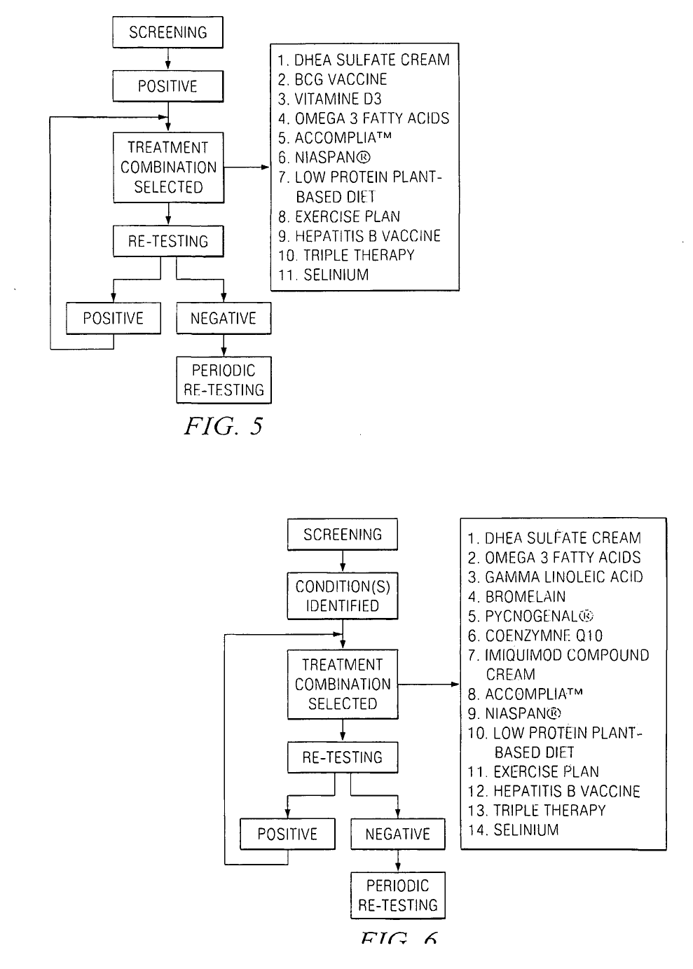

Referring now to FIGS. 5 and 6, The invention further comprises another treatment method for treating cancers and auto-immune diseases. Referring to FIG. 5, a method for treating cancers comprises a first step of screening according to traditional cancers screening methods well known to those skilled in the medical field and screening for breast cancer according to the methods shown in FIGS. 1, 2, and 4 and described herein in conjunction therewith. If the patient tests positive for cancer, a doctor determines a treatment combination for the patient, the treatment comprising one or more of the following treatment options:

(1) Topical administration of a about ¾ to 1 cc of about 50 mg DHEA sulfate cream. The DHEA sulfate cream is prepared by mixing a cream form of a hormone dehydroepiandrosterone (DHEA) with a commercially available non-steroid cream, the mixture resulting in a non-anabolic steroid cream which is applied topically for the purpose of attracting and activating killer-T Lymphocytes in order to treat cancer. A primary carrying agent for DHEA is a proprietary compound, Q-base cream. Q-base cream is available to compound pharmacies all over the United States and consists of the following formulation:
purified water, cyclomethicone, octyl stearate, arlacel 165 (PEG-100 stearate/glyceryl stearate), sorbitol, green tea extract (organic), stearyl alcohol, cyclopentasiloxane and PEG/PPG-18/18 dimethicone, gingko extract (organic), ginseng extract (organic), tocotrienols, vitamin E acetate, wheat germ oil, edetate disodium, xanthan gum, vitamin A palmitate, coenzyme Q10, methylchloroisothiazolinone/methylisothiazolin one.

The ratio of ingredients comprising the Q-base cream/DHEA mixture is between about 25 mg and about 50 mg of DHEA to about 1 cc of Q-base cream.

Alternatively, the carrying agent for the DHEA may comprise H-base cream. The formula for H-base cream is as follows:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The ratio of ingredients comprising the H-base cream/DHEA mixture is between about 25 mg and about 50 mg of DHEA to about 1 cc of H-base cream.

The DHEA cream mixture in either the Q-base cream/DHEA mixture or H-base cream/DHEA mixture is combined with a cream comprising sulfate, preferably chondroitin sulfate and/or glucosamine sulfate. One such commercially available cream is a deep tissue massage lotion produced by Lasting Touch™, “Lasting Touch Deep Tissue Therapy Massage Lotion.” In accordance with the present invention, the DHEA cream mixture, either Q-base or H-base, is mixed with the cream comprising sulfate at a 1:1 resulting in a DHEA sulfate cream for topical application;

(2) Administration of a BCG vaccine containing tumor necrosis factor (also called TNFa, cachexin, or cachetin) which stimulates T-cells that coordinate the immune system;

(3) Oral administration of about 400 IU to about 800 IU capsules once or twice daily of calcitriol or 1,25-dihydroxycholecalciferol, which is the active form of vitamin D3 found in the body. Vitamin D3 aids the body in absorption of calcium and phosphate from the kidneys and gastrointestinal tract;

(4) Oral administration of Omega 3 Fatty acids which have several enumerated health benefits, including, but not limited to increased blood circulation, reduction in blood triglyceride levels, protective effects against cardiovascular disease, and many other benefits well known to those skilled in the medical field. The preferable brand and dosage of Omega 3 fatty acids is Lovaza® brand fish oil pills, three to four 1 g capsule taken two times per day;

(5) Once daily oral administration of about 10 mg to about 20 mg of ACOMPLIA™ (rimonabant), a Cannabinoid receptor type 1 (CB1) blocker, taken once daily;

(6) Oral administration of a tablet of about 500 mg to about 1,000 mg of NIASPAN™ (niacin), an extended release form of niacin available from Abbot Laboratories®, which blocks the breakdown of fats and alters blood lipid levels. The NIASPAN™ (niacin) tablet is taken once daily at or near bedtime. If symptoms such as severe flushing and/or itching occur, the patient may also take a pain reliever such as acetaminophen and an antihistamine such as diphenhydramine in conjunction with the NIASPAN™ (niacin) tablet;

(7) Following a low protein, plant-based diet;

(8) Maintaining a directed exercise plan;

(9) Administration of a ¾ cc to about 1 cc RECOMBIVAX HB® (recombinant) Hepatitis B vaccine, administered once a month for three consecutive months and followed by a booster vaccine six months after the third consecutive vaccine; and

(10) Following a triple therapy treatment comprising a) FLAGYL® (metronidazole), a drug administered primarily for the treatment of infections caused by susceptible organisms, the FLAGYL® (metronidazole) administered both by topical administration of a cream and/or oral administration of one half to one of an about 250 mg to about 500 mg tablet twice daily, b) oral administration of one about 400 mg tablet of AVELOX® (moxifloxacin) taken once daily, a drug primarily used in the treatment of respiratory infections, and c) oral administration of one 20 mg to 40 mg tablet of NEXIUM® (Esomeprazole) once daily, a proton pump inhibitor used to reduce gastric acid secretions and also used in treatment combinations for Helicobacter pylori infections. The selection of, dosage, and frequency of any selected treatment is determined and adjusted by the patient's doctor according to the patient's body. The triple therapy comprising a combination of FLAGYL® (metronidazole), NEXIUM® (esomeprazole), and AVELOX® (moxifloxacin) may be administered beginning one month after starting the combination of treatments selected by the doctor and may be continued for two weeks thereafter.

The patient is thereafter re-tested periodically according to traditional cancer screening and diagnostic testing methods well known to those skilled in the medical field, including the blood CA 27,29 test mentioned hereinabove. Further treatment is directed as needed according to the outcome of the periodic testing. As the patient's symptoms and test results improve, testing may be performed at less frequent intervals such as yearly or bi-yearly to monitor the patient's continued health status.

Referring now to FIG. 6, a method for treating auto-immune diseases comprises a first step of screening and diagnosis according to traditional diagnostic methods for each individual disease well known to those skilled in the medical field. After a patient is diagnosed with one or more specific auto-immune diseases, the patient begins a combination treatment plan determined according to the patient's doctor, the combination treatment plan comprising one or more medications and treatments selected from the following medications and treatments: 1) topical administration of ¾ to about 1¼ cc daily of 50 mg of the DHEA sulfate cream mixture as described hereinabove; 2) oral administration of Omega 3 fatty acids, available in many brands and forms but preferably prescription omega-3-acid ethyl esters available from brand Lovaza®, taken once or twice daily in doses of about three to four 1 g capsules once daily oral administration of one tablet of about 300 mg to about 480 mg of gamma linoleic acid taken once daily, an essential fatty acid found in flax oil and Dihomo-gamma-linolenic acid (DGLA); 4) oral administration of three 500 mg capsules of bromelain taken two to three times daily, a mixture of sulfur-containing protein-digesting enzymes currently used for a vast array of medical conditions, available from Bio-Tech Pharmaceuticals™ under the name BROMASE™ (bromelain); 5) oral administration of about 75 mg to about 150 mg of an anti-oxidant such as Pycnogenol® (French maritime pine bark extract) available from Horphag® Research Corp., taken one to two times daily; 6) oral administration of one capsule of vitamin coenzyme Q-10 in strength of about 60 mg to about 120 mg taken once or twice daily; 7) topical application of one half to one packets of compounded imiquimod 5% cream, described hereinabove in conjunction with FIG. 2, which induces the production of interferon alpha once inside the human body, providing the needed interferon alpha not adequately produced by the patient's own body; 8) oral administration of one 10 mg to 20 mg tablet of ACOMPLIA™ (rimonabant) taken once daily, a Cannabinoid receptor type 1 (CB1) blocker; 9) Oral administration of about 500 mg to about 1000 mg of NIASPAN™ (niacin) taken at or around bedtime, an extended release form of niacin available from Abbot Laboratories®, which blocks the breakdown of fats and alters blood lipid levels, along with both a non-prescription pain reliever such as acetaminophen and an antihistamine such as diphenhydramine as necessary if side effects such as severe flushing and itching occur; 10) following a low protein, plant-based diet; 11) maintaining a directed exercise plan; 12) administration of a RECOMBIVAX HB® (recombinant) Hepatitis B vaccine, administered in doses of ¾ to 1 cc once a month for three consecutive months followed by a booster vaccine administered six months after the third vaccine; and 13) triple therapy treatment comprising a) FLAGYL® (metronidazole), a drug administered primarily for the treatment of infections caused by susceptible organisms, the FLAGYL® (metronidazole) administered both by topical administration of a cream and oral administration of one half to one tablet of about 250 mg to about 500 mg twice daily, b) oral administration of one half to one 400 mg tablet of AVELOX® (moxifloxacin) taken once daily, a drug primarily used in the treatment of respiratory infections, and c) oral administration of one about 20 mg to about 40 mg tablet of NEXIUM® (Esomeprazole) once or twice daily, a proton pump inhibitor used to reduce gastric acid secretions and also used in treatment combinations for Helicobacter pylori infections. The selection of, dosage, and frequency of any selected treatment is determined and adjusted by the patient's doctor according to the patient's body. The triple therapy comprising a combination of FLAGYL® (metronidazole), NEXIUM® (esomeprazole), and AVELOX® (moxifloxacin) may be administered beginning one month after starting the combination of treatments selected by the doctor and may be continued for two weeks thereafter.

The patient is thereafter tested periodically according to traditional screening and testing methods for each individual auto-immune disease well known to those skilled in the medical field. Further treatment is directed as needed according to the outcome of the periodic testing. As the patient's symptoms and test results improve, testing may be performed at less frequent intervals such as yearly or bi-yearly to monitor the patient's continued health and verify the disease is maintained or remains in remission.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of treating breast cancer in a patient comprising the steps of:
providing multiple treatments selected from the group consisting of oral administration of rimonabant, oral administration of calcitriol, intravenous administration of a recombinant Hepatitis B vaccine, directing a low protein diet, directing an exercise plan, transdermal administration of a cream comprising imiquimod 5% cream and H base cream, oral administration of a combination of tramadol and valacyclovir, and oral administration of omega 3 fatty acids;
selecting at least 2 of the provided multiple treatments from the group and prescribing the selected treatments for the patient;
performing a biopsy on at least one suspicious tissue sample taken from the patient; and
monitoring the results of the recombinant vaccines using a PET scan.

2. The method of treating duct cell breast cancer according to claim 1 wherein the results of the recombinant vaccines using a CAT scan.

3. The method of treating duct cell breast cancer according to claim 1 wherein rimonabant is administered once daily in a dose of about 10 mg to about 20 mg.

4. The method of treating duct cell breast cancer according to claim 1 wherein calcitriol is administered once daily in a dose of about 400 IU to about 800 IU.

5. The method of treating duct cell breast cancer according to claim 1 wherein calcitriol is administered twice daily in a dose of about 400 IU to about 800 IU.

6. The method of treating duct cell breast cancer according to claim 1 wherein the recombinant Hepatitis B vaccine is administered once a month for three consecutive months followed by a booster vaccine administered six months later.

7. The method of treating duct cell breast cancer according to claim 1 wherein tramadol is consumed in doses of about 50 mg to about 100 mg daily in conjunction with VALTREX® (valacyclovir) consumed twice daily in doses of about 500 mg tablets.

8. The method of treating duct cell breast cancer according to claim 1 wherein VALTREX® (valacyclovir) is consumed twice daily in doses of about 500 mg.

9. The method of treating duct cell breast cancer according to claim 1 wherein omega 3 fatty acids are administered three to four capsules of 1 g tablets of Lovaza® brand fish oil tablets are administered twice daily.

10. A method of treating cancer in a patient comprising the steps of:
providing multiple treatments selected from the group consisting of:
a. topical administration of a DHEA sulfate cream comprising a first cream comprising a mixture of Q-base cream and the steroid hormone DHEA prepared at a ratio of between about 25 mg and about 50 mg of DHEA to about 1 cc of Q-base cream and a second cream comprising sulfate, the first and second creams mixed at a ratio of about 1:1;
b. administration of a BCG vaccine;
c. oral administration of calcitriol;
d. oral administration of omega 3 fatty acids;
e. oral administration of rimonabant;
f. oral administration of extended release niacin;
g. directing a low protein diet;
h. directing an exercise plan;
i. oral administration of recombinant Hepatitis B vaccine;
j. administration of a triple therapy treatment comprising metronidizale, administered both topically and orally, oral administration of moxifloxacin, and oral administration of esomeprazole; and
selecting at least 2 of the provided multiple treatments from the group and prescribing the selected treatments for the patient.

11. The method of treating cancer according to claim 10 wherein the first cream comprising the DHEA sulfate cream is a mixture of H-base cream and the steroid hormone DHEA prepared at a ratio of between about 25 mg and about 50 mg of DHEA to about 1 cc of H-base cream.

12. The method of treating cancer according to claim 10 wherein the BCG vaccine is administered every three weeks.

13. The method of treating cancer according to claim 10 wherein calcitriol is administered once daily in a dose of about 400 IU to about 800 IU.

14. The method of treating cancer according to claim 10 wherein omega 3 fatty acids are administered in doses of three to four capsules of 1 g tablets of Lovaza® brand fish oil tablets administered twice daily.

15. The method of treating cancer according to claim 10 wherein rimonabant is administered once daily in a tablet of about 20 mg to about 40 mg.

16. The method of treating cancer according to claim 10 wherein extended release niacin is provided in the form of one tablet of NIASPAN™ (niacin) of about 500 mg to about 1.00 mg taken at or around bedtime.

17. The method of treating cancer according to claim 10 wherein the wherein the recombinant Hepatitis B vaccine is administered once a month for three consecutive months followed by a booster vaccine administered six months later.

18. The method of treating cancer according to claim 10 wherein the triple therapy comprises orally administering about 250 mg to about 500 mg of FLAGYL® (metronidazole) twice daily, orally administering about one 400 mg tablet of AVELOX® (moxifloxacin) once daily and orally administering one tablet of about 20 mg to about 40 mg of NEXIUM® (Esomeprazole) once daily.

19. A method of treating an auto-immune disease in a patient comprising the steps of:
providing multiple treatments selected from the group consisting of:
a. topical administration of a DHEA sulfate cream comprising a first cream comprising a mixture of Q-base cream and the steroid hormone DHEA prepared at a ratio of between about 25 mg and about 50 mg of DHEA to about 1 cc of Q-base cream and a second cream comprising sulfate, the first and second creams mixed at a ratio of about 1:1;
b. oral administration of omega 3 fatty acids;
c. oral administration of gamma linoleic acid;
d. oral administration of BROMASE®;
e. oral administration of PYCNOGEL® (French maritime pine bark extract);
f. oral administration of coenzyme Q10;
g. topical administration of compounded 5% imiquimod cream;
h. oral administration of rimonabant;
i. oral administration of extended release niacin;
j. directing a low protein diet;
k. directing an exercise plan;
l. oral administration of recombinant Hepatitis B vaccine;
m. administration of a triple therapy treatment comprising metronidizale, administered both topically and orally, oral administration of moxifloxacin, and oral administration of esomeprazole; and
selecting at least 2 of the provided multiple treatments from the group and prescribing the selected treatments for the patient.

20. The method of treating an auto-immune disease according to claim 21 wherein the first cream comprising the DHEA sulfate cream is a mixture of H-base cream and the steroid hormone DHEA prepared at a ratio of between about 2 mg and about 50 mg of DHEA to about 1 cc of H-base cream.

21. The method of treating cancer according to claim 21 wherein omega 3 fatty acids are administered in doses of three to four capsules of 1 g tablets of Lovaza® brand fish oil tablets administered twice daily.

22. The method of treating cancer according to claim 21 wherein gamma linoleic acid is administered twice daily in doses of one to two capsules of about 300 mg to about 480 mg.

23. The method of treating cancer according to claim 21 wherein three 500 mg capsules of BROMASE® are administered twice daily.

24. The method of treating cancer according to claim 21 wherein two capsules of about 75 mg to about 100 mg of PYCNOGEL® (French maritime pine bark extract) are administered twice daily.

25. The method of treating cancer according to claim 21 wherein one capsule of about 60 mg to about 120 mg of coenzyme Q10 is administered twice daily.

26. The method of treating cancer according to claim 21 wherein one tablet of about 10 mg to 20 mg of rimonabant is administered once daily.

27. The method of treating cancer according to claim 21 wherein the extended release niacin is provided in the form of one 500 mg tablet of NIASPAN™ (niacin) taken at or around bedtime.

28. The method of treating cancer according to claim 21 wherein the wherein the recombinant Hepatitis B vaccine is administered once a month for three consecutive months followed by a booster vaccine administered six months later.

29. The method of treating cancer according to claim 21 wherein the triple therapy comprises orally administering one half to one tablet of about 250 mg to about 500 mg of FLAGYL® (metronidazole) twice daily, orally administering one 400 mg tablet of AVELOX® (moxifloxacin) once daily and orally administering one tablet about 20 mg to about 40 mg of NEXIUM® (Esomeprazole) once daily.

30. The method of treating cancer according to claim 21 wherein the triple therapy comprises transdermally administering a FLAGYL® (metronidazole) cream twice daily, orally administering one 400 mg tablet of AVELOX® (moxifloxacin) once daily and orally administering one tablet of about 20 mg to about 40 mg of NEXIUM® (Esomeprazole) once daily.
